(19) United States
(12) Patent Application Publication
(10) Pub. No.: US 2026/0210850 A1
(43) Pub. Date: Jul. 23, 2026

(54) DUAL COMB GENERATING APPARATUS AND SPECTROSCOPY SYSTEM INCLUDING THE SAME

(71) Applicant: ELECTRONICS AND TELECOMMUNICATIONS RESEARCH INSTITUTE, Daejeon (KR)

(72) Inventor: Minje SONG, Daejeon (KR)

(21) Appl. No.: 19/444,981

(22) Filed: Jan. 9, 2026

(30) Foreign Application Priority Data
Jan. 9, 2025 (KR) 10-2025-0003651
Jan. 7, 2026 (KR) 10-2026-0002519

(51) Int. Cl. G01N 21/39; G01N 33/00; G02F 1/01

(52) U.S. Cl. CPC G01N 21/39; G01N 33/0027; G02F 1/0121; G02F 2203/56

(57) ABSTRACT

A dual comb generating apparatus and a spectroscopy system including the same is provided. The dual comb generating apparatus includes a continuous-wave laser configured to output a continuous optical signal, a first RF source configured to generate a first RF signal, a second RF source configured to generate a second RF signal, a hybrid coupler configured to impart a phase difference to the first and second RF signals, and an electro-optic modulation module configured to generate two optical frequency combs with mutually different repetition rates and center frequencies from the optical signal, by using the first and second RF signals output from the hybrid coupler as driving signals.

### CROSS REFERENCE TO RELATED APPLICATIONS

This application claims priority under 35 U.S.C. § 119 to Korean Patent Application No. 10-2025-0003651 filed on Jan. 9, 2025 and Korean Patent Application No. 10-2026-0002519 filed on Jan. 7, 2026, in the Korean Intellectual Property Office, the disclosures of which are incorporated by reference herein in their entireties.

### TECHNICAL FIELD

The present invention relates to a dual comb generating apparatus and a spectroscopy system including the same, and more particularly, to a dual comb generating apparatus and a spectroscopy system including the same that can generate a plurality of optical frequency combs with different repetition rates and center frequencies.

### BACKGROUND ART

An optical frequency comb is an optical source that provides constant and precise frequency intervals over a wide spectral range, and is utilized in various fields such as coherent optical communication systems, coherent detection, microwave photonics, and high-precision spectroscopy. Particularly in the field of spectroscopy, the optical frequency comb plays a key role in various application areas requiring high sensitivity and high selectivity, such as environmental monitoring, biological material analysis, and chemical component detection.

Dual-comb spectroscopy (DCS), which uses two optical frequency combs, is attracting attention as a key technology for implementing a real-time, high-resolution spectroscopy system because it can analyze broadband spectral information in real time with high resolution without temporal or spatial movement.

Conventional methods for implementing a dual comb have been proposed, utilizing micro-ring resonator-based micro-combs, mode-locked lasers, and electro-optic frequency combs. Generally, the micro-comb method is advantageous in terms of miniaturization, but in a real-time high-resolution measurement environment, high stability and precise repetition rate adjustment are required, so mode-locked laser or electro-optic frequency comb-based methods are preferred. In particular, to respond to various environmental changes in real time, flexible adjustability is required, and in this respect, the importance of the electro-optic frequency comb is further emphasized. However, conventional dual-comb implementation methods require separate control (PID controllers, laser locking systems, etc.) for stabilization and synchronization to ensure mutual stability, which ultimately increases system complexity.

The background technology of the present invention is disclosed in Korean Laid-Open Patent Publication No. 10-2693498 (published on Aug. 5, 2024).

### SUMMARY OF THE INVENTION

### Problem to be Solved

An object according to an aspect of the present invention is to provide a dual comb generating apparatus and a spectroscopy system including the same, which may adjust the repetition rate and center frequency of an optical frequency comb through the generation of an electro-optic modulation-based optical frequency comb.

Furthermore, an object according to an aspect of the present invention is to provide a dual comb generating apparatus and a spectroscopy system including the same, which may improve the mutual stability between optical frequency combs without a separate synchronization device by simultaneously generating a plurality of optical frequency combs with different repetition rates and center frequencies within a single apparatus using a single laser.

However, the technical problems to be solved by the present invention are not limited to the problems mentioned above, and other unmentioned problems can be clearly understood by those skilled in the art from the description of the invention below.

### Solution to Problem

A dual comb generating apparatus according to an embodiment of the present invention is characterized by including: a continuous-wave laser configured to output a continuous optical signal; a first RF source configured to generate a first RF signal; a second RF source configured to generate a second RF signal; a hybrid coupler configured to impart a phase difference to the first and second RF signals; and an electro-optic modulation module configured to generate two optical frequency combs with mutually different repetition rates and center frequencies from the optical signal, by using the first and second RF signals output from the hybrid coupler as driving signals.

In the present invention, the first RF source may be configured to make the frequency of the first RF signal variable, and the second RF source may be configured to make the frequency of the second RF signal variable.

In the present invention, the first and second RF signals may have different frequencies from each other.

In the present invention, the hybrid coupler is characterized by imparting a 90° phase difference to the first and second RF signals.

In the present invention, the electro-optic modulation module is characterized by including: an optical intensity modulation module configured to intensity-modulate the optical signal output from the continuous-wave laser, by using the first and second RF signals output from the hybrid coupler as respective driving signals; and an optical phase modulation module configured to phase-modulate the optical signal output from the optical intensity modulation module, by using the first and second RF signals output from the hybrid coupler as respective driving signals.

In the present invention, the optical intensity modulation module is characterized by including: a first optical intensity modulator configured to generate a first intensity-modulated signal by intensity-modulating the optical signal according to the first RF signal output from the hybrid coupler; and a second optical intensity modulator configured to generate a second intensity-modulated signal by intensity-modulating the optical signal according to the second RF signal output from the hybrid coupler.

In the present invention, the optical phase modulation module is characterized by including: a first optical phase modulator configured to phase-modulate the first intensity-modulated signal according to the first RF signal output from the hybrid coupler; and a second optical phase modulator configured to phase-modulate the second intensity-modulated signal according to the second RF signal output from the hybrid coupler.

The present invention is further characterized by including a nonlinear stage configured to expand the bandwidth of the optical frequency combs.

A spectroscopy system according to an embodiment of the present invention is characterized by including: a dual comb generating apparatus configured to generate two optical frequency combs with mutually different repetition rates and center frequencies from a continuous optical signal using first and second RF signals as driving signals, and to irradiate a target sample with the two optical frequency combs; an optical filter module configured to receive the optical signal in which the two optical frequency combs have been superimposed after interacting with the target sample, and to separate it by wavelength; and a detection module configured to convert the optical signal output from the optical filter module into an electrical signal and detect it.

In the present invention, the dual comb generating apparatus is characterized by including: a continuous-wave laser configured to output a continuous optical signal; a first RF source configured to generate a first RF signal; a second RF source configured to generate a second RF signal; a hybrid coupler configured to impart a phase difference to the first and second RF signals; and an electro-optic modulation module configured to generate two optical frequency combs with mutually different repetition rates and center frequencies from the optical signal, by using the first and second RF signals output from the hybrid coupler as driving signals.

In the present invention, the first RF source may be configured to make the frequency of the first RF signal variable, and the second RF source may be configured to make the frequency of the second RF signal variable.

In the present invention, the first and second RF signals may have different frequencies from each other.

In the present invention, the hybrid coupler is characterized by imparting a 90° phase difference to the first and second RF signals.

In the present invention, the electro-optic modulation module is characterized by including: an optical intensity modulation module configured to intensity-modulate the optical signal output from the continuous-wave laser, by using the first and second RF signals output from the hybrid coupler as respective driving signals; and an optical phase modulation module configured to phase-modulate the optical signal output from the optical intensity modulation module, by using the first and second RF signals output from the hybrid coupler as respective driving signals.

In the present invention, the optical intensity modulation module is characterized by including: a first optical intensity modulator configured to generate a first intensity-modulated signal by intensity-modulating the optical signal according to the first RF signal output from the hybrid coupler; and a second optical intensity modulator configured to generate a second intensity-modulated signal by intensity-modulating the optical signal according to the second RF signal output from the hybrid coupler.

In the present invention, the optical phase modulation module is characterized by including: a first optical phase modulator configured to phase-modulate the first intensity-modulated signal according to the first RF signal output from the hybrid coupler; and a second optical phase modulator configured to phase-modulate the second intensity-modulated signal according to the second RF signal output from the hybrid coupler.

In the present invention, the dual comb generating apparatus is further characterized by including a nonlinear stage configured to expand the bandwidth of the optical frequency combs.

In the present invention, the optical filter module is characterized by including: a first optical filter configured to extract an optical signal of a first wavelength band; and a second optical filter configured to extract an optical signal of a second wavelength band, which is higher than the first wavelength band.

In the present invention, the detection module is characterized by including: a first photodiode connected to the first optical filter to convert the optical signal of the first wavelength band into an electrical signal; and a second photodiode connected to the second optical filter to convert the optical signal of the second wavelength band into an electrical signal.

### Advantageous Effects of the Invention

According to the present invention, the repetition rate and center frequency of an optical frequency comb can be adjusted through the generation of an electro-optic modulation-based optical frequency comb.

Furthermore, according to the present invention, by simultaneously generating a plurality of optical frequency combs with different repetition rates and center frequencies within a single device using a single laser, the mutual stability between the optical frequency combs can be improved without a separate synchronization device.

Furthermore, according to the present invention, by omitting the synchronization device required for synchronization between optical frequency combs, the structure of the dual comb generating apparatus is simplified, and the maintenance and operational convenience of the dual comb generating apparatus are improved.

Furthermore, according to the present invention, a high-precision spectroscopy system capable of responding to environmental changes in real-time can be implemented by using a dual comb generating apparatus that can adjust the center wavelength and repetition rate of the optical frequency comb.

However, the effects that can be obtained through the present invention are not limited to the effects mentioned above, and other technical effects not mentioned can be clearly understood by those skilled in the art from the description of the invention below.

### BRIEF DESCRIPTION OF DRAWINGS

FIG. 1 is a block diagram showing a dual comb generating apparatus according to an embodiment of the present invention.

FIG. 2 is an exemplary diagram for explaining a hybrid coupler.

FIG. 3 is an exemplary diagram for explaining the first and second RF signals to which a phase difference has been imparted.

FIG. 4 is a block diagram showing a spectroscopy system according to an embodiment of the present invention.

FIG. 5 is a block diagram showing a spectroscopy system according to another embodiment of the present invention.

### DETAILED DESCRIPTION OF THE INVENTION

Hereinafter, preferred embodiments of the present invention will be described in detail with reference to the accompanying drawings. Prior to this, the terms or words used in this specification and claims should not be interpreted in a conventional or dictionary sense, but should be interpreted in a meaning and concept that are consistent with the technical idea of the present invention, based on the principle that the inventor can appropriately define the concept of the term to explain his own invention in the best way. Therefore, the configurations shown in the embodiments and drawings described in this specification are merely some of the most preferred embodiments of the present invention and do not represent all of the technical ideas of the present invention, so it should be understood that there can be various equivalents and modifications that can replace them at the time of filing this application. Also, when the terms “comprise, include” and/or “comprising, including” are used in this specification, they specify the presence of stated shapes, numbers, steps, operations, members, elements and/or groups thereof, and do not preclude the presence or addition of one or more other shapes, numbers, operations, members, elements and/or groups thereof. Also, when describing embodiments of the present invention, “can” or “may” can include “one or more embodiments of the present invention.”

Furthermore, to aid in the understanding of the invention, the accompanying drawings are not drawn to actual scale, and the dimensions of some components may be exaggerated. Also, for the same components in different embodiments, the same reference numerals may be assigned.

The statement that two compared objects are ‘identical’ means ‘substantially identical’. Therefore, ‘substantially identical’ may include cases with small deviations considered by those skilled in the art, for example, deviations within 5%. Also, the statement that a certain parameter is uniform in a given region means that it is uniform from an average perspective.

Although terms such as first, second, etc., may be used to describe various components, these components should not be limited by these terms. These terms are only used to distinguish one component from another, and unless there is a contrary statement, a first component could be a second component.

Throughout the specification, unless there is a contrary statement, each component may be singular or plural.

The statement that any configuration is placed “on (or under)” a component or “above (or below)” a component means not only that the configuration is placed in contact with the top (or bottom) surface of the component, but also that another component may be interposed between the component and the configuration placed on (or under) it.

Also, when a component is stated to be “connected,” “coupled,” or “joined” to another component, it may be directly connected or joined, but it should be understood that another component may be “interposed” between the respective components, or that the respective components may be “connected,” “coupled,” or “joined” through another component. Also, when a part is electrically coupled to another part, this includes not only cases where they are directly connected, but also cases where they are connected with another device interposed between them.

Throughout the specification, when “A and/or B” is stated, it means A, B, or both A and B, unless there is a contrary statement. That is, “and/or” includes any and all combinations of one or more of the associated listed items. When “C to D” is stated, it means C or more and D or less, unless there is a contrary statement.

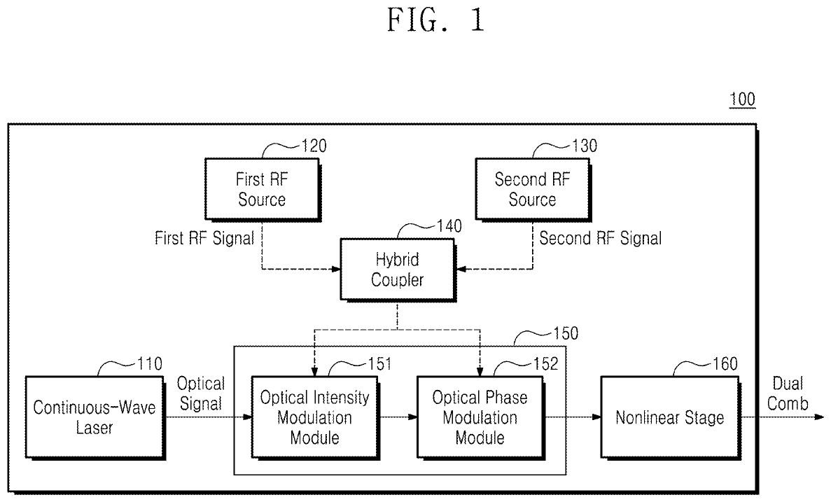

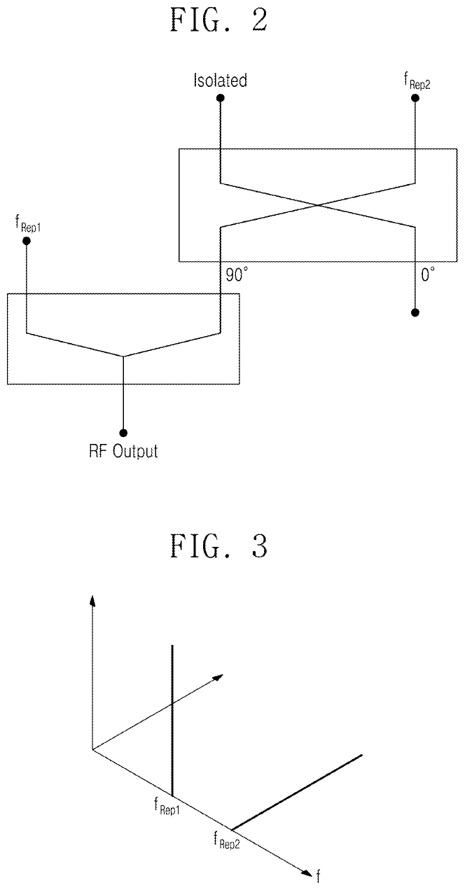

FIG. 1 is a block diagram showing a dual comb generating apparatus according to an embodiment of the present invention, FIG. 2 is an exemplary diagram for explaining a hybrid coupler, and FIG. 3 is an exemplary diagram for explaining the first and second RF signals to which a phase difference has been imparted.

Referring to FIG. 1, a dual comb generating apparatus 100 according to an embodiment of the present invention may include a continuous-wave laser 110, a first RF source 120, a second RF source 130, a hybrid coupler 140, an electro-optic modulation module 150, and a nonlinear stage 160. The dual comb generating apparatus 100 according to an embodiment of the present invention may include various other components in addition to the components shown in FIG. 1, and may not include some of the components shown in FIG. 1. For example, the dual comb generating apparatus 100 according to an embodiment of the present invention may not include the nonlinear stage 160.

The continuous-wave laser 110 may output a continuous optical signal (continuous wave). The continuous-wave laser 110 may output an optical signal of a single wavelength. The optical signal output from the continuous-wave laser 110 can be applied to the electro-optic modulation module 150. The wavelength of the optical signal output through the continuous-wave laser 110 may be changed. That is, the continuous-wave laser 110 may be a wavelength-tunable continuous-wave laser 110 configured to change the wavelength of the output laser. For example, the continuous-wave laser 110 may be a DFB (distributed feedback) laser with a wavelength band of 1550 nm and a linewidth smaller than 100 kHz.

The first RF source 120 may generate a first RF signal. The first RF source 120 may generate an RF signal having a predetermined frequency. The first RF source 120 may change the frequency and phase of the first RF signal. The first RF source 120 may be implemented as an oscillator or a frequency synthesizer. In various embodiments, the first RF source 120 may include both an oscillator and a frequency synthesizer. However, the configuration of the first RF source 120 is not limited to the aforementioned embodiment, and various devices capable of generating an RF (radio frequency) signal may be used as the first RF source 120. The first RF signal generated from the first RF source 120 may be applied to the hybrid coupler 140.

The second RF source 130 may generate a second RF signal. The second RF source 130 may generate an RF signal having a predetermined frequency. The second RF signal may be an RF signal with a frequency different from that of the first RF signal. The second RF source 130 may change the frequency and phase of the second RF signal. The second RF source 130 may be implemented as an oscillator or a frequency synthesizer. In various embodiments, the second RF source 130 may include both an oscillator and a frequency synthesizer. However, the structure of the second RF source 130 is not limited to the aforementioned embodiment, and various devices capable of generating an RF signal may be used as the second RF source 130. The second RF signal generated from the second RF source 130 may be applied to the hybrid coupler 140.

The hybrid coupler 140 may impart a phase difference to the first and second RF signals. The hybrid coupler 140 may impart a 90° phase difference to the first and second signals. The first and second RF signals to which a phase difference has been imparted by the hybrid coupler 140 may be applied to the electro-optic modulation module 150. As shown in FIG. 2, a 90° phase difference may be imparted between the first and second RF signals f_{Rep1}, f_{Rep2} by the hybrid coupler 140, and as a result, a first RF signal f_{Rep1} and a second RF signal f_{Rep2} with a 90° phase difference from the first RF signal f_{Rep1} may be output from the hybrid coupler 140. FIG. 3 is a diagram showing the frequencies of the first and second RF signals f_{Rep1}, f_{Rep2} output from the hybrid coupler 140 on the x-axis, and the phases of the first and second RF signals f_{Rep1}, f_{Rep2} output from the hybrid coupler 140 as angles with respect to the xy-plane.

The electro-optic modulation module 150 may generate two optical frequency combs (e.g., a dual comb) with mutually different repetition rates and center frequencies (or phases) from the optical signal output from the continuous-wave laser 110 by using the first and second RF signals output from the hybrid coupler (the first and second RF signals to which a phase difference has been imparted) as driving signals. In this embodiment, the repetition rate may mean the spacing between frequency components in the optical frequency comb.

In various embodiments, the electro-optic modulation module 150 may be configured to generate two or more optical frequency combs. Hereinafter, for convenience of explanation, it will be assumed that the electro-optic modulation module 150 generates two optical frequency combs.

The electro-optic modulation module 150 may generate an optical frequency comb by an electro-optic modulation method. The electro-optic modulation module 150 may include an optical intensity modulation module 151 and an optical phase modulation module 152.

The optical intensity modulation module 151 may intensity-modulate the optical signal output from the continuous-wave laser 110 by using the first and second RF signals output from the hybrid coupler 140 as respective driving signals. The optical intensity modulation module 151 may include a first optical intensity modulator and a second optical intensity modulator. The optical intensity modulation module 151 may include a plurality of intensity modulators corresponding to the number of optical frequency combs to be generated.

The first optical intensity modulator may generate a first intensity-modulated signal by intensity-modulating the optical signal output from the continuous-wave laser 110 according to the first RF signal output from the hybrid coupler 140. The first intensity-modulated signal generated by the first optical intensity modulator may be applied to the first optical phase modulator described below.

The second optical intensity modulator may generate a second intensity-modulated signal by intensity-modulating the optical signal output from the continuous-wave laser 110 according to the second RF signal output from the hybrid coupler 140. The second intensity-modulated signal generated by the second optical intensity modulator may be applied to the second optical phase modulator described below.

The optical phase modulation module 152 may phase-modulate the optical signals output from the optical intensity modulation module 151 by using the first and second RF signals output from the hybrid coupler 140 as respective driving signals. The optical phase modulation module 152 may include a first optical phase modulator and a second optical phase modulator. The optical phase modulation module 152 may include a plurality of phase modulators corresponding to the number of optical frequency combs to be generated.

The first optical phase modulator may phase-modulate the first intensity-modulated signal generated by the first optical intensity modulator according to the first RF signal output from the hybrid coupler 140. As the optical signal intensity-modulated by the first optical intensity modulator is phase-modulated by the first optical phase modulator, a first optical frequency comb is generated.

The second optical phase modulator may phase-modulate the second intensity-modulated signal generated by the second optical intensity modulator according to the second RF signal output from the hybrid coupler 140. As the optical signal intensity-modulated by the second optical intensity modulator is phase-modulated by the second optical phase modulator, a second optical frequency comb is generated.

In various embodiments, the optical intensity modulation module 151 may include only one optical intensity modulator. In this case, the optical intensity modulator may intensity-modulate the optical signal output from the continuous-wave laser 110 using either the first RF signal or the second RF signal output from the hybrid coupler 140 as a driving signal, and the intensity-modulated optical signal may be split and applied to each of the plurality of optical phase modulators included in the optical phase modulation module 152.

In various embodiments, the optical phase modulation module 152 may include only one optical phase modulator. In this case, the optical phase modulator may phase-modulate the optical signal that has been intensity-modulated by the optical intensity modulator, by using a superimposed RF signal of the first and second RF signals output from the hybrid coupler 140 as a driving signal, thereby generating an optical signal that simultaneously includes optical frequency comb components with mutually different repetition rates.

The nonlinear stage 160 may expand the bandwidth of each of the two optical frequency combs generated by the electro-optic modulation module 150. The nonlinear stage 160 may be configured to induce nonlinear interactions between optical signals by inputting the optical frequency combs into a nonlinear medium. Such nonlinear interactions may include at least one of self-phase modulation, cross-phase modulation, or four-wave mixing.

By the nonlinear stage 160, the phase of each frequency component constituting the optical frequency comb is nonlinearly modulated, thereby generating new frequency components on both sides of the spectrum of the existing optical frequency comb. Accordingly, the optical frequency comb generated by the electro-optic modulation module 150 may have its spectral bandwidth expanded while maintaining its center frequency. The nonlinear stage 160 may include at least one of a nonlinear fiber, an integrated nonlinear waveguide, and a nonlinear crystal. By expanding the bandwidth through the nonlinear stage 160, the spectral resolution and measurable wavelength range of the optical frequency comb may be improved.

For example, if the continuous-wave laser 110 is set to output an optical signal in the 1500 nm wavelength band, the first RF source 120 is set to generate an RF signal with a frequency of 10 GHz, and the second RF source 130 is set to generate an RF signal with a frequency of 10.01 GHz, two optical frequency combs with a repetition rate difference of about 10 MHz may be generated by the dual comb generating apparatus 100.

According to the present embodiment, by adjusting the frequencies of the RF signals generated by the first RF source 120 and the second RF source 130, the repetition-rate difference between the optical frequency combs may be adjusted over a broad range from several kHz to several tens of GHz. Such broad tunability may provide significant technical advantages in spectroscopy environments involving various analytes. In addition, according to the present embodiment, by simultaneously generating, within a single apparatus using a single laser, a plurality of optical frequency combs having different repetition rates and center frequencies, the phase relationship among the optical frequency combs may be stably maintained, thereby enabling a high signal-to-noise ratio SNR to be secured.

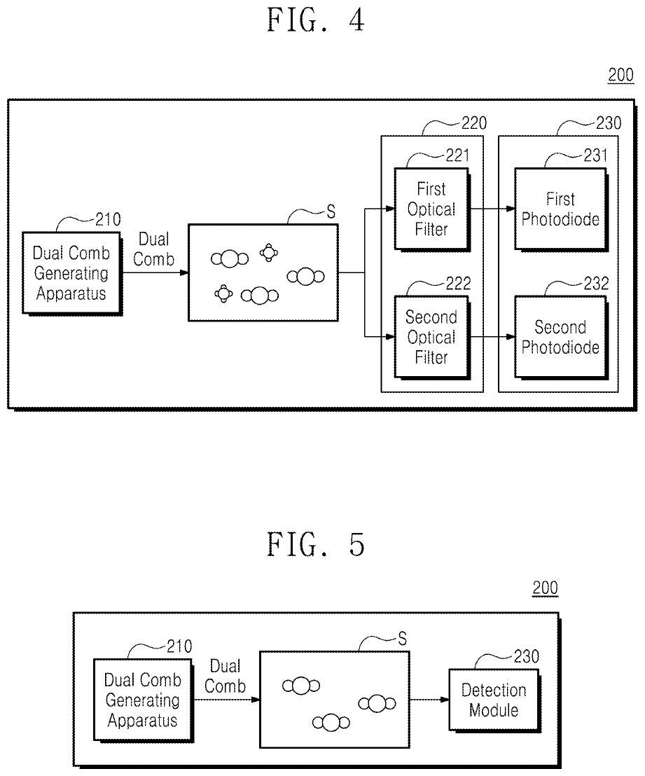

FIG. 4 is a block diagram showing a spectroscopy system according to an embodiment of the present invention.

Referring to FIG. 4, a spectroscopy system 200 according to an embodiment of the present invention may include a dual comb generating apparatus 210, an optical filter module 220, and a detection module 230. The spectroscopy system 200 according to an embodiment of the present invention may include various other components in addition to the components shown in FIG. 4, and may not include some of the components shown in FIG. 4.

The dual comb generating apparatus 210 may generate two optical frequency combs with mutually different repetition rates and center frequencies from a continuous optical signal using first and second RF signals as driving signals, and may irradiate a target sample S with the two generated optical frequency combs. The target sample S may be provided in a measurement environment such as a gas chamber, and the dual comb generating apparatus 210 may be configured such that the optical frequency combs generated by it pass through the measurement environment.

The dual comb generating apparatus 210 may include a continuous-wave laser configured to output a continuous optical signal, a first RF source configured to generate a first RF signal, a second RF source configured to generate a second RF signal, a hybrid coupler configured to impart a phase difference to the first and second RF signals, and an electro-optic modulation module configured to generate two optical frequency combs with mutually different repetition rates and center frequencies from the optical signal output from the continuous-wave laser by using the first and second RF signals output from the hybrid coupler as driving signals. The dual comb generating apparatus 210 may further include a nonlinear stage configured to expand the bandwidth of the optical frequency combs. Since the configuration and operation of the dual comb generating apparatus 210 have already been described above, a redundant explanation thereof will be omitted.

The optical filter module 220 may receive the optical signal in which the two optical frequency combs have been superimposed after interacting with the target sample S, and may separate it by wavelength. The optical frequency combs that have passed through the target sample S, or the optical frequency combs reflected from the target sample S, may be received by the optical filter module 220. The optical filter module 220 may include a first optical filter 221 and a second optical filter 222.

The first optical filter 221 may extract an optical signal of a first wavelength band. The first optical filter 221 may selectively pass an optical signal belonging to the first wavelength band. The second optical filter 222 may extract an optical signal of a second wavelength band. The second optical filter 222 may selectively pass an optical signal belonging to the second wavelength band. The second wavelength band may be a higher wavelength band than the first wavelength band. However, the number of filters included in the optical filter module 220 and the wavelength band detected by each filter are not limited to the aforementioned embodiment, and the number of filters included in the optical filter module 220 and the wavelength band of the optical signal extracted by each filter may be changed according to the designer's intention. The first and second optical filters 221, 222 may be OBPFs (Optical Band-Pass Filters).

The detection module 230 may convert the optical signal output from the optical filter module 220 into an electrical signal (RF signal) and detect it. The detection module 230 may include a first photodiode 231 and a second photodiode 232.

The first photodiode 231 may be connected to the first optical filter 221 to convert the optical signal of the first wavelength band output from the first optical filter 221 into an electrical signal (first electrical signal). The second photodiode 232 may be connected to the second optical filter 222 to convert the optical signal of the second wavelength band output from the second optical filter 222 into an electrical signal (second electrical signal).

The first and second photodiodes 231, 232 may generate a frequency down-converted RF signal. The first and second photodiodes 231, 232 nonlinearly detect the superimposed optical frequency combs to generate an RF signal corresponding to the difference between the optical frequency components, and accordingly, frequency down-conversion may be performed. By analyzing the frequency down-converted RF signal generated by the first and second photodiodes 231, 232, the characteristics (absorption spectrum, phase change information) of the target sample S may be measured with high resolution.

In fields such as environmental monitoring or chemical analysis, high-resolution, high-precision spectroscopy is essential for detecting trace gas concentration changes in real time. The spectroscopy system 200 according to an embodiment of the present invention may meet these demands and enable rapid concentration measurement and analysis.

FIG. 5 is a block diagram showing a spectroscopy system according to another embodiment of the present invention.

As shown in FIG. 5, the spectroscopy system 200 according to an embodiment of the present invention may be composed of a dual comb generating apparatus 210 and a detection module 230. That is, the spectroscopy system 200 according to an embodiment of the present invention may not include the optical filter module 220, and in this case, the superimposed optical signal of the two optical frequency combs that has interacted with the target sample S may be received by the detection module 230.

As such, according to the present invention, the repetition rate and center frequency of an optical frequency comb may be adjusted through the generation of an electro-optic modulation-based optical frequency comb. Furthermore, according to the present invention, by simultaneously generating a plurality of optical frequency combs with different repetition rates and center frequencies within a single apparatus using a single laser, the mutual stability between the optical frequency combs may be improved without a separate synchronization device. Furthermore, according to the present invention, by omitting the synchronization device required for synchronization between optical frequency combs, the structure of the dual comb generating apparatus is simplified, and the maintenance and operational convenience of the dual comb generating apparatus are improved. Furthermore, according to the present invention, a high-precision spectroscopy system capable of responding to environmental changes in real time may be implemented by using a dual comb generating apparatus that may adjust the center wavelength and repetition rate of the optical frequency comb.

The terms “part” and “module” used in this specification may include a unit implemented as hardware, software, or firmware, and for example, may be used interchangeably with terms such as logic, logical block, component, or circuit. A “part” and a “module” can be an integrally configured component or a minimum unit or a part thereof that performs one or more functions. For example, according to an embodiment, a “part” and a “module” can be implemented in the form of an Application-Specific Integrated Circuit (ASIC).

The implementations described in this specification may be implemented, for example, as a method or process, a device, a software program, a data stream, or a signal. Even if discussed only in the context of a single form of implementation (e.g., discussed only as a method), the implementation of the discussed feature can also be implemented in other forms (e.g., a device or a program). A device may be implemented with appropriate hardware, software, and firmware. A method may be implemented in a device, such as, for example, a computer, a microprocessor, an integrated circuit, or a programmable logic device, which is generally referred to as a processing device. A processor also includes communication devices such as computers, cell phones, personal digital assistants (“PDAs”), and other devices that facilitate communication of information between end-users.

Although the present invention has been described by limited embodiments and drawings, the present invention is not limited thereto, and it is a matter of course that various modifications and variations are possible within the technical idea of the present invention and the equivalent scope of the claims to be described below by those of ordinary skill in the art to which the present invention pertains.

### DESCRIPTION OF REFERENCE NUMERALS

100: Dual comb generating apparatus
110: Continuous wave laser
120: First RF source
130: Second RF source
140: Hybrid coupler
150: Electro-optic modulation module
151: Optical intensity modulation module
152: Optical phase modulation module
160: Nonlinear stage
200: Spectroscopy system
210: Dual comb generating apparatus
220: Optical filter module
221: First optical filter
222: Second optical filter
230: Detection module
231: First photodiode
232: Second photodiode

What is claimed is:

1. A dual comb generating apparatus, comprising:
a continuous-wave laser configured to output a continuous optical signal;
a first RF source configured to generate a first RF signal;
a second RF source configured to generate a second RF signal;
a hybrid coupler configured to impart a phase difference to the first and second RF signals; and
an electro-optic modulation module configured to generate two optical frequency combs with mutually different repetition rates and center frequencies from the optical signal, by using the first and second RF signals output from the hybrid coupler as driving signals.

2. The dual comb generating apparatus of claim 1, wherein the first RF source is configured to vary a frequency of the first RF signal, and
wherein the second RF source is configured to vary a frequency of the second RF signal.

3. The dual comb generating apparatus of claim 1, wherein the first and second RF signals have different frequencies from each other.

4. The dual comb generating apparatus of claim 1, wherein the hybrid coupler imparts a 90° phase difference to the first and second RF signals.

5. The dual comb generating apparatus of claim 1, wherein the electro-optic modulation module comprises:
an optical intensity modulation module configured to intensity-modulate the optical signal output from the continuous-wave laser, by using the first and second RF signals output from the hybrid coupler as respective driving signals; and
an optical phase modulation module configured to phase-modulate the optical signal output from the optical intensity modulation module, by using the first and second RF signals output from the hybrid coupler as respective driving signals.

6. The dual comb generating apparatus of claim 5, wherein the optical intensity modulation module comprises:
a first optical intensity modulator configured to generate a first intensity-modulated signal by intensity-modulating the optical signal according to the first RF signal output from the hybrid coupler; and
a second optical intensity modulator configured to generate a second intensity-modulated signal by intensity-modulating the optical signal according to the second RF signal output from the hybrid coupler.

7. The dual comb generating apparatus of claim 6, wherein the optical phase modulation module comprises:
a first optical phase modulator configured to phase-modulate the first intensity-modulated signal according to the first RF signal output from the hybrid coupler; and
a second optical phase modulator configured to phase-modulate the second intensity-modulated signal according to the second RF signal output from the hybrid coupler.

8. The dual comb generating apparatus of claim 1, further comprising a nonlinear stage configured to expand the bandwidth of the optical frequency combs.

9. A spectroscopy system, comprising:
a dual comb generating apparatus configured to generate two optical frequency combs with mutually different repetition rates and center frequencies from a continuous optical signal using first and second RF signals as driving signals, and irradiate a target sample with the two optical frequency combs;
an optical filter module configured to receive an optical signal in which the two optical frequency combs are superimposed after interacting with the target sample, and to separate the optical signal by wavelength; and
a detection module configured to convert the optical signal output from the optical filter module into an electrical signal and detect the electrical signal.

10. The spectroscopy system of claim 9, wherein the dual comb generating apparatus comprises:
a continuous-wave laser configured to output a continuous optical signal; a first RF source configured to generate a first RF signal; a second RF source configured to generate a second RF signal;
a hybrid coupler configured to impart a phase difference to the first and second RF signals; and
an electro-optic modulation module configured to generate two optical frequency combs with mutually different repetition rates and center frequencies from the optical signal, by using the first and second RF signals output from the hybrid coupler as driving signals.

11. The spectroscopy system of claim 10, wherein the first RF source is configured to make the frequency of the first RF signal variable, and the second RF source is configured to make the frequency of the second RF signal variable.

12. The spectroscopy system of claim 10, wherein the first and second RF signals have different frequencies from each other.

13. The spectroscopy system of claim 10, wherein the hybrid coupler imparts a 90° phase difference to the first and second RF signals

14. The spectroscopy system of claim 10, wherein the electro-optic modulation module comprises:
an optical intensity modulation module configured to intensity-modulate the optical signal output from the continuous-wave laser, by using the first and second RF signals output from the hybrid coupler as respective driving signals; and
an optical phase modulation module configured to phase-modulate the optical signal output from the optical intensity modulation module, by using the first and second RF signals output from the hybrid coupler as respective driving signals.

15. The spectroscopy system of claim 14, wherein the optical intensity modulation module comprises:
a first optical intensity modulator configured to generate a first intensity-modulated signal by intensity-modulating the optical signal according to the first RF signal output from the hybrid coupler; and
a second optical intensity modulator configured to generate a second intensity-modulated signal by intensity-modulating the optical signal according to the second RF signal output from the hybrid coupler.

16. The spectroscopy system of claim 15, wherein the optical phase modulation module comprises:
a first optical phase modulator configured to phase-modulate the first intensity-modulated signal according to the first RF signal output from the hybrid coupler; and
a second optical phase modulator configured to phase-modulate the second intensity-modulated signal according to the second RF signal output from the hybrid coupler.

17. The spectroscopy system of claim 10, wherein the dual comb generating apparatus further comprises a nonlinear stage configured to expand the bandwidth of the optical frequency combs.

18. The spectroscopy system of claim 9, wherein the optical filter module comprises:
a first optical filter configured to extract an optical signal of a first wavelength band; and
a second optical filter configured to extract an optical signal of a second wavelength band, which is higher than the first wavelength band.

19. The spectroscopy system of claim 18, wherein the detection module comprises:
a first photodiode connected to the first optical filter to convert the optical signal of the first wavelength band into an electrical signal; and
a second photodiode connected to the second optical filter to convert the optical signal of the second wavelength band into an electrical signal.
